(19) United States
(12) Patent Application Publication
(10) Pub. No.: US 2025/0262265 A1
(43) Pub. Date: Aug. 21, 2025

(54) ANTI-ARGINASE COMPOSITIONS OF NUTRIENTS, BOTANICALS, PHYTOCHEMICALS AND BIOACTIVE COMPOUNDS IN HUMANS

(71) Applicant: Partner. Co., Midvale, UT (US)

(72) Inventors: Brett Justin West, Cedar Hills, UT (US); Fernando Antonio Swartz, Bournemouth (GB)

(73) Assignee: Partner. Co., Midvale, UT (US)

(21) Appl. No.: 19/046,657

(22) Filed: Feb. 6, 2025

Related U.S. Application Data
(60) Provisional application No. 63/554,579, filed on Feb. 16, 2024.

(51) Int. Cl. A61K 36/746; A61K 31/12; A61K 31/197; A61K 31/198; A61K 31/455; A61K 31/714; A61K 33/04; A61K 33/06; A61K 33/24; A61K 33/30; A61K 36/185; A61K 36/45; A61K 36/73; A61K 36/87; A61K 36/9066

(52) U.S. Cl. CPC A61K 36/746; A61K 31/12; A61K 31/197; A61K 31/198; A61K 31/455; A61K 31/714; A61K 33/04; A61K 33/06; A61K 33/24; A61K 33/30; A61K 36/185; A61K 36/45; A61K 36/73; A61K 36/87; A61K 36/9066

(57) ABSTRACT

The present invention relates to a formulation providing health promoting and anti-aging benefits that may include amino acids, vitamins, mineral co-factors, antioxidants, carotenoids, and phytonutrients from herbal sources, plant products or their derivatives. When combined, these ingredients provide increased and unexpected synergistic effects that that inhibit arginase activity and enhance circulating L-arginine and nitric oxide levels.

### TECHNICAL FIELD

The present invention relates to a health promoting and anti-aging dietary supplement. More specifically, the present invention relates to methods and compositions that provide improved health benefits using a combination of micronutrients including amino acids, vitamins, mineral co-factors, antioxidants, carotenoids, and phytonutrients from herbal sources, plant products or their derivatives, as well as other nutrients adapted for human consumption, that inhibit arginase activity and enhance circulating L-arginine and nitric oxide levels.

### SUMMARY

Embodiments of the present invention relate to an anti-aging and health promoting dietary supplement. The present invention provides for a series of complex formulations and combinations wherein said formulations are designed for supporting human health. The rationale for the multiple compositions and the dosing schedule is explained below.

In one embodiment, the formulation comprises Acerola Fruit (Malpighia glabra) Extract, Blackberry (Rubus spp.) Fruit, Blueberry (Vaccinium spp.) Fruit Extract, Turmeric (Curcuma longa) Root Extract (containing curcumin), Grape (Vitis vinifera) Seed Extract, and Noni (Morinda citrifolia) Fruit. The formulation further comprises an amino acid comprising L-Arginine, L-Citrulline, beta-Alanine, and L-Lysine. The formulation also comprises a carotenoid. The formulation also comprises gamma-aminobutyric acid (GABA).

The formulation further comprises L-Arginine, L-Citrulline, L-Lysine, beta-alanine, Vitamin B3 as Niacin, Vitamin B3 as Niacinamide, Vitamin B6, Vitamin B12, chromium, magnesium, selenium, zinc, a carotenoid, and gamma-aminobutyric acid.

In one example embodiment, the formulation comprises, by weight of formulation: L-Arginine, L-Citrulline, and L-Lysine in an amount between 120 and 6000 mg; beta-alanine in an amount between 5 and 250 mg; Vitamin B3 as Niacin in an amount between 1 and 100 mg; Vitamin B3 as Niacinamide in an amount between 5 and 1000 mg; Vitamin B6 in an amount between 0.5 and 25 mg; Vitamin B12 in an amount between 0.001 and 0.05 mg; chromium in an amount between 0.005 and 0.25 mg; magnesium in an amount between 12 and 600 mg; selenium in an amount between 0.01 and 30 mg; zinc in an amount between 1.5 and 100 mg; beta-carotene in an amount between 0.05 and 20 mg; a carotenoid other than beta-carotene, in an amount between 0.05 and 50 mg; Vaccinium corymbosum fruit extract in an amount between 1.5 and 75 mg; Curcuma longa root extract which comprises curcumin in an amount between 1.5 and 125 mg; Rubus fruticosus fruit in an amount between 1.0 and 75 mg; Vitis vinifera seed extract in an amount between 1.5 and 125 mg; Malpighia glabra fruit in an amount between 0.5 and 50 mg; gamma-aminobutyric acid in an amount between 50 and 2500 mg; and Morinda citrifolia fruit in an amount between 5 and 500 mg.

The formulation further comprises, by weight of formulation: L-Arginine in an amount equal to 1200 mg; L-Citrulline in an amount equal to 1218.27 mg; L-Lysine in an amount equal to 1218.27 mg; beta-alanine in an amount equal to 50 mg; Vitamin B3 as Niacin in an amount equal to 5.05 mg; Vitamin B3 as Niacinamide in an amount equal to 20.3 mg; Vitamin B6 in an amount equal to 6.25 mg; Vitamin B12 in an amount equal to 0.01 mg; chromium in an amount equal to 0.27 mg; magnesium in an amount equal to 250 mg; selenium in an amount equal to 20 mg; zinc in an amount equal to 50 mg; beta-carotene in an amount equal to 0.434 mg; a carotenoid other than beta-carotene, in an amount equal to 1.8 mg; Vaccinium corymbosum fruit extract in an amount equal to 25 mg; Curcuma longa root extract which comprises curcumin in an amount equal to 25 mg; Rubus fruticosus fruit in an amount equal to 15 mg; Vitis vinifera seed extract in an amount equal to 25 mg; Malpighia glabra fruit in an amount equal to 5 mg; gamma-aminobutyric acid in an amount equal to 505.05 mg; and Morinda citrifolia fruit in an amount equal to 65 mg.

In an alternative example embodiment, the formulation comprises, by weight of formulation: L-Arginine, L-Citrulline, and L-Lysine in an amount between 120 and 6000 mg; beta-alanine in an amount between 5 and 250 mg; Vitamin B3 as Niacinamide in an amount between 5 and 1000 mg; Vitamin B6 in an amount between 0.5 and 25 mg; Vitamin B12 in an amount between 0.001 and 1.5 mg; chromium in an amount between 0.005 and 0.5 mg; magnesium in an amount between 12 and 600 mg; selenium in an amount between 0.01 and 50 mg; zinc in an amount between 1.5 and 125 mg; beta-carotene in an amount between 0.05 and 20 mg; a carotenoid other than beta-carotene, in an amount between 0.05 and 50 mg; Vaccinium corymbosum fruit extract in an amount between 1.5 and 75 mg; Curcuma longa root extract which comprises curcumin in an amount between 1.5 and 125 mg; Rubus fruticosus fruit in an amount between 1.0 and 75 mg; Vitis vinifera seed extract in an amount between 1.5 and 125 mg; Malpighia glabra fruit in an amount between 0.5 and 50 mg; gamma-aminobutyric acid in an amount between 50 and 2500 mg; and Morinda citrifolia fruit in an amount between 5 and 500 mg.

The formulation further comprises, by weight of formulation: L-Arginine in an amount equal to 1400 mg; L-Citrulline in an amount equal to 1015.22 mg; L-Lysine in an amount equal to 1218.27 mg; beta-alanine in an amount equal to 50.76 mg; Vitamin B3 as Niacinamide in an amount equal to 25.38 mg; Vitamin B6 in an amount equal to 6.25 mg; Vitamin B12 in an amount equal to 0.36 mg; chromium in an amount equal to 0.15 mg; magnesium in an amount equal to 250 mg; selenium in an amount equal to 50 mg; zinc in an amount equal to 125 mg; beta-carotene in an amount equal to 0.434 mg; a carotenoid other than beta-carotene, in an amount equal to 1.8 mg; Vaccinium corymbosum fruit extract in an amount equal to 25 mg; Curcuma longa root extract which comprises curcumin in an amount equal to 25 mg; Rubus fruticosus fruit in an amount equal to 15 mg; Vitis vinifera seed extract in an amount equal to 25 mg; Malpighia glabra fruit in an amount equal to 5 mg; gamma-aminobutyric acid in an amount equal to 505.05 mg; and Morinda citrifolia fruit in an amount equal to 65 mg.

### BRIEF DESCRIPTION OF THE FIGURES

Features, aspects, and advantages of the present invention are better understood when the following detailed description of the invention is read with reference to the accompanying figures, in which:

FIG. 1 is a graph showing measured inhibition and expected inhibition between botanicals individually and the combination of botanicals.

### BACKGROUND

Arginase is an L-arginine metabolizing enzyme. It is involved in some fundamental biological mechanisms of the human body and catalyzes the hydrolysis of L-arginine to L-ornithine and urea. However, arginase activity increases in a tissue-dependent manner during aging, leading to many of the undesired physiological changes associated with the aging process. Further, elevated arginase expression and/or activity leads to biological and biochemical dysregulation and is a risk factor for several diseases such as cancer, cardiovascular diseases, neurodegenerative diseases. It may also contribute to increased mortality.

Arginase competes with nitric oxide synthase (NOS) for L-arginine as a substrate. A primary biological function of NOS is the production of nitric oxide (NO) from L-arginine. With elevated arginase expression and activity, L-arginine depletion leads to NOS decoupling and the subsequent production of reactive oxygen species (ROS) which subsequently cause cellular and tissue damage. This contributes to the aging process.

Arginase may also contribute to the aging process by causing changes in cell survival (autophagy, mTOR or IGF-1) pathways. This is especially true for the aging of the vasculature. Further, asymmetric dimethylarginine (ADMA) increases with biological age. ADMA competes with L-arginine as a substrate for NOS further exacerbating the decoupling of NO production and subsequent formation of ROS such as superoxide anion radicals (SAR). In fact, L-arginine: ADMA ratios in the body decline with advancing age. A corresponding decline in NO: SAR ratios also occur with increased age. NO provides significant cellular antioxidant protection. Therefore, the decoupling of NO production by NOS from L-arginine is doubly problematic as it leads to elevated oxidative stress, inflammation, and degenerative changes that are hallmarks of aging.

Mitigating these age-related adverse effects by supplementation with a combination of dietary ingredients which reduce elevated arginase activity, increase levels of bioavailable L-arginine and its metabolite, NO, is a safe and effective anti-aging strategy. Such supplementation will also provide health benefits among younger adults as well. This is due to the role of NO in blood vessel function, blood pressure regulation, circulation, immune system function, and antioxidant protection. Additionally, NO plays an important role in the biological signaling pathways that lead to growth hormone release from the pituitary.

### DETAILED DESCRIPTION

### Product Formulations

The present invention will now be described more fully hereinafter with reference to the accompanying pictures in which exemplary embodiments of the invention are shown. However, the invention may be embodied in many different forms and should not be construed as limited to the representative embodiments set forth herein. The exemplary embodiments are provided so that this disclosure will be both thorough and complete and will fully convey the scope of the invention and enable one of ordinary skill in the art to make, use, and practice the invention. Although the following description provides embodiments of the invention by way of example, it is envisioned that other embodiments may perform similar functions and/or achieve similar results. Any and all such equivalent embodiments and examples are within the scope of the present invention.

Relative terms such as lower or bottom; upper or top; upward, outward, or downward; forward or backward; and vertical or horizontal may be used herein to describe one element's relationship to another element illustrated in the figures. It will be understood that relative terms are intended to encompass different orientations in addition to the orientation depicted in the drawings. Relative terminology, such as “substantially” or “about,” describe the specified materials, steps, parameters, or ranges as well as those that do not materially affect the basic and novel characteristics of the claimed inventions as whole (as would be appreciated by one of ordinary skill in the art).

This invention provides novel formulations that are derived from five entirely separate base ingredient categories:
a) Amino Acids
b) Vitamins and Mineral Co-factors
c) Antioxidant and Carotenoids
d) Herbals, Plant Derivatives and Phytonutrients
e) Other Nutrients

Those of skill in the art will appreciate that the inventive formulations disclosed in this Application can include varying combinations and concentrations of the base ingredient categories and may omit one or more of the base ingredient categories while achieving the desired benefits. Moreover, each base ingredient category can itself include combinations of compounds. For instance, the base ingredient category “amino acids” can include multiple amino acids within a single formulation embodiment.

The combinations of base ingredient categories included in the complete formulation, and the combinations of compounds within each base ingredient category, provide further enhancement to the unique nature of the present formulations. Thereby, individuals may consume different ingredient levels within the broad dosage ranges provided. Consumption may also entail none of the ingredients from a particular category, all the ingredients in a category or partial combinations thereof.

Example formulations are shown below with dosing for each ingredient. Each example formulation also includes dosing ranges for each ingredient. The doses listed are intended to be the complete daily dosage. For commercial application, this total dose may be divided in partial servings to be taken at different times of the day depending on the product line format (capsule, liquid, powder, etc.).

### Standard Formulation, Adults 18 Years and Older, Consisting Essentially of:

### Base Formula

| Ingredient | Amount | Unit |
|---|---|---|
| L-Arginine Base | 1200 | mg |
| L-Citrulline | 1218.27 | mg |
| L-Lysine HCL | 1218.27 | mg |
| Beta-Alanine | 50 | mg |
| Vitamin B3 (as Niacin) | 5.05 | mg |
| Vitamin B3 (as Niacinamide) | 20.3 | mg |
| Vitamin B6 (from Pyridoxine HCL) | 6.25 | mg |
| Vitamin B12 (as Methylcobalamin) | 0.01 | mg |
| Chromium chloride | 0.27 | mg |
| Magnesium amino acid chelate | 250 | mg |
| L-selenomethionine | 20 | mg |
| Zinc citrate | 50 | mg |
| Beta-carotene | 0.434 | mg |
| Carotenoids (other than beta-carotene but | 1.8 | mg |
| inclusive of lutein, zeaxanthin, etc.) | | |
| Blueberry (Vaccinium spp.) Fruit Extract | 25 | mg |
| Turmeric (Curcuma longa) Root Extract | 25 | mg |
| Blackberry (Rubus spp.) fruit powder | 15 | mg |
| Grape (Vitis vinifera) Seed Extract | 25 | mg |
| Acerola (Malpighia glabra) fruit powder | 5 | mg |
| GABA (gamma-aminobutyric acid) | 505.05 | mg |
| Noni (Morinda citrifolia) Fruit | 65 | mg |

### Range Formula

Amount in mg

| Ingredient | Low | High |
|---|---|---|
| L-Arginine | 120 | 6000 |
| L-Citrulline | 120 | 6000 |
| L-Lysine HCL | 120 | 6000 |
| Beta-Alanine | 5 | 250 |
| Vitamin B3 (as Niacin) | 1 | 100 |
| Vitamin B3 (as Niacinamide) | 5 | 1000 |
| Vitamin B6 (from Pyridoxine HCL) | 0.5 | 25 |
| Vitamin B12 (as Methylcobalamin) | 0.001 | 0.05 |
| Chromium | 0.005 | 0.25 |
| Magnesium | 12 | 600 |
| Selenium | 0.01 | 30 |
| Zinc | 1.5 | 100 |
| Beta-carotene | 0.05 | 20 |
| Carotenoids (other than beta-carotene but | 0.05 | 50 |
| lutein, zeaxanthin, etc.) | | |
| Blueberry (Vaccinium spp.) Fruit Extract | 1.5 | 75 |
| Turmeric (Curcuma longa) Root Extract | 1.5 | 125 |
| (containing curcumin) | | |
| Blackberry (Rubus spp.) fruit powder | 1.0 | 75 |
| Grape (Vitis vinifera) Seed Extract | 1.5 | 125 |
| Acerola (Malpighia glabra) fruit powder | 0.5 | 50 |
| GABA (gamma-aminobutyric acid) | 50 | 2500 |
| Noni (Morinda citrifolia) Fruit | 5 | 500 |

### Alternate Formula 1

| Ingredient | Amount | Unit |
|---|---|---|
| L-Arginine Base | 1400 | mg |
| L-Citrulline | 1015.22 | mg |
| L-Lysine HCL | 1218.27 | mg |
| L-Alanine | 50.76 | mg |
| Vitamin B3 (as Niacinamide) | 25.38 | mg |
| Vitamin B6 (from Pyridoxine HCL) | 6.25 | mg |
| Vitamin B12 (as Cyanocobalamin) | 0.36 | mg |
| Chromium chloride (within high chromium | 0.15 | mg |
| yeast or selenium yeast) | | |
| Magnesium amino acid chelate | 250 | mg |
| L-Selenomethionine | 50 | mg |
| Zinc gluconate | 125 | mg |
| Beta-carotene | 0.434 | mg |
| Carotenoids (other than beta-carotene but | 1.8 | mg |
| inclusive of lutein, zeaxanthin, etc.) | | |
| Blueberry (Vaccinium spp.) Fruit Extract | 25 | mg |
| Turmeric (Curcuma longa) Root Extract | 25 | mg |
| Blackberry (Rubus spp.) fruit powder | 15 | mg |
| Grape (Vitis vinifera) Seed Extract | 25 | mg |
| Acerola (Malpighia glabra) fruit powder | 5 | mg |
| GABA (gamma-aminobutyric acid) | 505.05 | mg |
| Noni (Morinda citrifolia) Fruit | 65 | mg |

### Alternative Range Formula 1

Amount in mg

| Ingredient | Low | High |
|---|---|---|
| L-Arginine | 120 | 6000 |
| L-Citrulline | 120 | 6000 |
| L-Lysine HCL | 120 | 6000 |
| Beta-Alanine | 5 | 250 |
| Vitamin B3 (as Niacinamide) | 5 | 1000 |
| Vitamin B6 (from Pyridoxine HCL) | 0.5 | 25 |
| Vitamin B12 (as Cyanocobalamin) | 0.001 | 1.5 |
| Chromium | 0.005 | 0.5 |
| Magnesium | 12 | 600 |
| Selenium | 0.01 | 50 |
| Zinc | 1.5 | 125 |
| Beta-carotene | 0.05 | 20 |
| Carotenoids (other than beta-carotene but | 0.05 | 50 |
| lutein, zeaxanthin, etc.) | | |
| Blueberry (Vaccinium spp.) Fruit Extract | 1.5 | 75 |
| Turmeric (Curcuma longa) Root Extract | 1.5 | 125 |
| (containing curcumin) | | |
| Blackberry (Rubus spp.) fruit powder | 1.0 | 75 |
| Grape (Vitis vinifera) Seed Extract | 1.5 | 125 |
| Acerola (Malpighia glabra) fruit powder | 0.5 | 50 |
| GABA (gamma-aminobutyric acid) | 50 | 2500 |
| Noni (Morinda citrifolia) Fruit | 5 | 500 |

### Alternate Formula 2

| Ingredient | Amount | Unit |
|---|---|---|
| L-Arginine Base | 1200 | mg |
| L-Citrulline | 1218.27 | mg |
| L-Lysine HCL | 1218.27 | mg |
| Beta-Alanine | 50 | mg |
| Vitamin B3 (as Niacin) | 1.08 | mg |
| Vitamin B6 (from Pyridoxine HCL) | 0.111 | mg |
| Vitamin B12 (as Cyanocobalamin) | 0.003 | mg |
| Magnesium amino acid chelate | 3.0 | mg |
| Zinc citrate | 15 | mg |
| Beta-carotene | 0.434 | mg |
| Carotenoids (other than beta-carotene but | 1.8 | mg |
| inclusive of lutein, zeaxanthin, etc.) | | |
| Blueberry (Vaccinium spp.) Fruit Extract | 30 | mg |
| Turmeric (Curcuma longa) Root Extract | 25 | mg |
| Blackberry (Rubus spp.) fruit powder | 15 | mg |
| Grape (Vitis vinifera) Seed Extract | 25 | mg |
| Acerola (Malpighia glabra) fruit powder | 5 | mg |
| Mangosteen (Garcinia mangostana L.) fruit | 30 | mg |
| extract | | |
| GABA (gamma-aminobutyric acid) | 505.05 | mg |
| Noni (Morinda citrifolia) Fruit | 65 | mg |

### Alternative Range Formula 2

Amount in mg

| Ingredient | Low | High |
|---|---|---|
| L-Arginine | 120 | 6000 |
| L-Citrulline | 120 | 6000 |
| L-Lysine HCL | 120 | 6000 |
| Beta-Alanine | 5 | 250 |
| Vitamin B3 (as Niacin) | 0.1 | 20 |
| Vitamin B6 (from Pyridoxine HCL) | 0.01 | 5 |
| Vitamin B12 (as Methylcobalamin) | 0.001 | 0.5 |
| Magnesium | 0.5 | 15 |
| Zinc | 1.5 | 75 |
| Beta-carotene | 0.05 | 1.5 |
| Carotenoids (other than beta-carotene but | 0.05 | 10 |
| lutein, zeaxanthin, etc.) | | |
| Blueberry (Vaccinium spp.) Fruit Extract | 1.5 | 75 |
| Turmeric (Curcuma longa) Root Extract | 2.5 | 125 |
| (containing curcumin) | | |
| Blackberry (Rubus spp.) fruit powder | 1.5 | 75 |
| Grape (Vitis vinifera) Seed Extract | 2.5 | 125 |
| Acerola (Malpighia glabra) fruit powder | 1.5 | 75 |
| Mangosteen (Garcinia mangostana L.) fruit | 0.01 | 35 |
| extract | | |
| GABA (gamma-aminobutyric acid) | 50 | 2500 |
| Noni (Morinda citrifolia) Fruit | 10 | 500 |

For individuals utilizing the “Standard Base” formulation, it is intended that they consume:
a) about four or more of the seven ingredients in the “Amino Acids” category,
b) the “Vitamins and Mineral Co-factors” category is optional, especially for individuals who are already consuming a multivitamin/mineral preparation
c) about two or more of the four ingredients in the “Antioxidants and Carotenoids” category
d) about three or more of the six ingredients in the “Herbals, Plant Derivatives and Phytonutrients” category, and
e) optionally the one ingredient in the “Other Nutrient” category

### Category and Ingredient Rationale

This section will discuss the scientific rationale for the use of the components of this invention in the major ingredient categories.

### Amino Acids

This category of agents is critical because of their biological characteristics that are of particular value in anti-aging formulations, especially since they constitute the largest component in body tissues (besides water). Amino acids may be essential (needed from the diet), non-essential (body can manufacture them) or conditional (body cannot produce them during stress or illness). The current invention contains all three types. Chains of amino acids make up peptides which are bioactive compounds that have beneficial metabolic effects. They also boost growth hormone levels as well as substances that maintain optimal pH balance. For clarity, unbranched chains of several peptides are known as polypeptides, and when longer than approximately 50 amino acids, the chains are designated as a protein. A bioactive protein is a “straight” polypeptide chain that is folded and optically arranged into a three-dimensional biologically functional configuration. Besides protein formation, these critical “building blocks” are essential for neurotransmitter function, immune system integrity, energy production, antioxidant defense, and nitric oxide-related molecular signaling for physiological processes.

L-arginine is an amino acid and the direct substrate for the family of nitric oxide synthases and is involved in metabolic processes of immune cells, including T-cell growth and lymphocyte function. As expected, L-arginine also increases nitric oxide production with the resultant promotion of vascular dilation as well as reduced LDL-cholesterol levels and platelet and white blood cell adhesion. It is further anticipated that muscle strength and recovery will be improved and provide ergogenic benefit. These expectations have been demonstrated in human studies where exertion-related oxygen consumption was reduced and exercise tolerance enhanced. In addition, oxidative damage can be decreased, immune function increased and organ function improved in experimental models. Of note, L-arginine and L-citrulline may be synergistic in these effects. L-citrulline is also included in this invention and is described in a subsequent section. With relevance to aging, L-arginine and L-lysine, alone and in combination, are anti-inflammatory and may benefit bone health.

The amino acid beta-alanine can be produced by the body (non-essential), is not involved in protein accretion but is the only naturally occurring beta-amino acid. Beta-alanine provides its greatest benefits by enhancing synthesis of the peptide carnosine and providing adjunctive effects to L-arginine.

Carnosine is a precursor to nitric oxide synthase generation and subsequent nitric oxide production. It may improve cognition and optimize muscular acid-base balance thereby increasing strength, mass, and endurance while decreasing fatigue. As noted previously, arginine is critical in nitric oxide production, energy metabolism, and immune function as well as reducing inflammation and harmful low-density lipoprotein profiles.

As implied above, L-citrulline is closely related to the metabolism of L-arginine. Since L-citrulline is not subject to hepatic degradation, it is metabolized into arginine and can enhance those levels effectively and play a role in protein synthesis and nitric oxide metabolism. The effect of increased nitric oxide on human performance has been extensively evaluated. Performance during exertion and recovery are positively influenced by L-citrulline in clinical trials of healthy males and athletes. This may have implications to preserving functional capacity as individuals age. In addition, the vascular effects of this amino acid have therapeutic effects in heart disease and cold exposure stress. The benefits also extend to the elderly in both clinical and experimental situations. Finally, L-citrulline exhibits beneficial influences in ischemic conditions and liver damage.

The use of the essential amino acid L-lysine as a food additive has been well characterized. It has several critical functions including anti-viral effects, support of collagen production, reduction of anxiety, and beneficial metabolic effects. The balance between L-lysine and its partner L-arginine is also significant since the latter amino acid enhances immune function through antibody development. Lysine also improves the efficacy of L-arginine in promoting human growth hormone release as well as for the production of NO. L-lysine is an arginase inhibitor, which further shunts L-arginine metabolism towards nitric oxide production by nitric oxide synthase.

### Vitamins and Mineral Co-Factors

Vitamin B3 or niacin is a water-soluble vitamin that exists in the acid (nicotinic acid) and amide (nicotinamide) form, each with different functions. This B vitamin is a component of key co-enzymes necessary for cellular metabolism and energy production as well as having a role as an antioxidant, in cell signaling and DNA synthesis and repair. The acid form of niacin improves lipid profiles and provides anti-inflammatory effects, both of which are vascular protective. The amide form of vitamin B3 is efficacious in photoprotection and chemoprevention from skin disease as well as osteoarthritis, two conditions particularly related to the aging process.

The most important functions of vitamin B6, pyridoxine, are related to the health of the nervous and immune systems. Specifically, this vitamin is involved in the biosynthesis of neurotransmitters thereby impacting cognitive development. Deficiency is associated with several clinical conditions resulting in low plasma concentrations. While adequate levels have not definitively shown effect in cancer, there may be some benefit in cardiovascular disease and neurocognitive domains.

Vitamin B12 is a water-soluble vitamin that is necessary for neurological function, DNA synthesis, and red cell formation as well as acting as an important enzymatic co-factor. As with other B vitamins, deficiency syndromes can show a wide spectrum of symptoms. Older individuals are especially affected. Because of its cognitive effects, vitamin B12 continues to be assessed in dementias. The enhanced consumption of L-arginine to produce NO induces a greater demand for B vitamins such as B6 and B12. This is because an important nitric oxide synthase cofactor, tetrahydrobiopterin, requires these vitamins for its biosynthesis.

The chemical element chromium has widespread applications including in human health. The trivalent ionic form is thought to be an essential nutrient because of its impact on lipid and glucose metabolism that are known to be altered with aging. This trace element has a mechanistic role in the action of insulin thereby affecting protein, carbohydrate and fat metabolism. The chromium content in foods is generally low so supplementation is a reasonable strategy and recommended adequate intakes have been established. Qualified health claims for chromium have been allowed in the U.S. and Canada as a factor in the maintenance of good health.

Magnesium holds a prominent position among essential elements that function as required co-factors for critical enzymatic reactions, biochemical function, and metabolic pathways. Its supplementation is generally safe and is an effective therapeutic intervention in many clinical situations. It may also have a role as an electrolyte. This mineral has been shown to be beneficial in regard to decreasing inflammatory responses and reducing complications in high-risk surgery.

Selenium is a co-factor of glutathione peroxidase, and Se-glutathione peroxidase also acts as an antioxidant. This mineral also decreases harmful effects of pro-inflammatory cytokines and is radio-protective. Recent experimental studies have shown that selenium may protect against DNA damage and inhibit tumor progression. It is also involved in the activity of superoxide dismutase providing further cellular defense against reactive oxygen species. Therefore, selenium supplementation at adequate but safe doses is essential for promoting antioxidant enzyme systems and reducing potential oxidative stress.

As has been emphasized in this section, trace elements play an important role in human health. Zinc is an essential micronutrient in this regard and functions as a biological catalyst and regulator with critical effects in oxidative stress, immune function, cellular integrity, and the aging process. In fact, since zinc levels decline with age, there are broad international efforts to ascertain appropriate supplementation guidelines. In this population, zinc is vital to maintenance of DNA repair and genomic stability. Zinc enhances superoxide dismutase activity and also provides a protective antioxidant function against peripheral neuropathy in diabetic animal models. Several chronic diseases related to oxidative stress and inflammation are associated with inadequate zinc levels.

### Antioxidants and Carotenoids

Intracellular antioxidants such as glutathione are important to the defense of the immune system as well as relevant to neurologic pathophysiology. Glutathione can catabolize anions and hydrogen peroxide and is a potent intracellular protective agent against excess free radical damage. However, since it is hydrolyzed in the intestine and generally ineffective when consumed orally, the formulations disclosed herein take advantage of alternative anti-aging strategies against oxidative damage including, but not limited to, NO production.

Although natural-source mixed carotenoids usually contain at least four carotene configurations (alpha, beta, gamma, delta), beta-carotene is the most common and dominant form. In the body, beta-carotene is a vitamer (similar molecular structure) and precursor of vitamin A. This carotenoid is an essential antioxidant that decreases free radical-induced oxidative stress. Vitamin A is critical for induction of beneficial cellular differentiation while beta-carotene increases the expression of the connexin gene, which encodes for a gap junction protein that is necessary in maintaining normal cellular phenotypes. It is also more effective in quenching oxygen-derived free radicals. Both these antioxidants protect cellular components in lipid and high oxygen environments but only the natural form of beta-carotene can effectively decrease radiation-induced cellular abnormalities. Beta-carotene, in combination with other antioxidants, is an effective radioprotectant and can address ischemia and reperfusion injury, biological damages relevant to aging.

Lutein is another dietary carotenoid that demonstrates antioxidant properties in humans with related health benefits. It functions as a strong antioxidant in vivo and reduces oxidative stress in individuals with low antioxidant status subjected to hostile environments. This substance has a unique characteristic in that it can induce increases in physical activity in sedentary persons and is also neuroprotective against transient brain ischemia. Relative to the current invention, multiple antioxidants are virtually always more effective than the individual agents themselves, such as noted in tumor cell growth inhibition.

Zeaxanthin is another effective dietary antioxidant. It is the pigment that gives bell peppers, corn, saffron, and many other plants and microbes their characteristic color. Zeaxanthin is one of the two major carotenoids contained within the retina of the eye. High blood lutein and zeaxanthin levels are significantly associated with a decrease in the risk of age-related nuclear cataracts.

### Botanical Ingredients—Herbals, Plant Derivatives and Phytonutrients

Botanical ingredients include species of blueberry fruits (genus Vaccinium) that contain polyphenols and anthocyanidin molecules which are powerful antioxidant compounds. These phytochemicals are readily soluble and bioavailable in many forms. Their bioactivity in terms of free radical scavenging and anti-inflammatory effect have been well documented. As an anti-aging strategy, the interplay between inflammation and obesity may provide another intervention target for blueberry fruits. Recent research demonstrates cardiometabolic-related improvement in lipid status, vascular function, and nitric oxide vasodilatory bioactivity. In addition, this phytonutrient has shown potential efficacy as an anti-cancer radiosensitizer. Of particular importance has been the emergence of blueberry polyphenols and anthocyanins as neuroprotective agents. Experimental and clinical studies have documented memory enhancement and neurocognitive improvement. Recent animal model investigations have elucidated likely mechanisms that are responsible for the observed neuroplasticity.

Curcumin is a phytonutrient abundantly present in the spice, turmeric, and demonstrates a wide spectrum of therapeutic properties. Its potential application to lung protection from hazardous exposures has been described. Immune enhancement with antibacterial activity is another potential benefit. Curcumin has important impact on cognitive decline through its antioxidant and anti-inflammatory actions as well as its contribution to neural plasticity and repair, hippocampal neurogenesis, and amyloid beta protein plaque binding. This polyphenolic agent decreases muscular pain and reduces inflammation and post-exercise muscle damage in human subjects. New highly absorbable formats may also benefit bone health. It improves in vivo neural functioning and protects against oxidative stress and brain ischemia-reperfusion injury. Curcumin has also shown benefits against oxidative damage, and in combination with resveratrol, demonstrates unique radioprotective qualities on normal tissues.

Grape seeds and extracts thereof protect against ultraviolet radiation via their phenolic compounds, the proanthocyanidins. Grape juice polyphenols and resveratrol improve antioxidant capacity and diminish oxidative stress. These compounds protect neurocognitive function in older adults and spatial memory and performance in younger adults. Resveratrol is the predominant polyphenolic compound derived from the skin of grapes and other berries and is also neuroprotective as a single agent. It has importance in environmental exposure as shown by its protective effect against the oxidative stress caused by cigarette smoke. In addition, there are number of studies that document its protective antioxidant effect against radiation damage.

In an adjunctive manner, the current formulations may utilize compounds chemically related to resveratrol such as pterostilbene. Pterostilbene is a naturally derived primary antioxidant component of blueberries with greater oral absorption and, through lipophilic function, greater bioavailability than resveratrol. It has demonstrated antioxidant, anti-inflammatory, biological and anticarcinogenic properties in experimental and in vivo models. However, while resveratrol is ‘Generally Recognized as Safe” (GRAS) for chronic use, pterostilbene's safety and efficacy has not been clinically evaluated long enough for full assessment or GRAS status designation.

Morinda citrifolia, commonly known as noni, possesses a wide range of biological activities that are beneficial to human health. Human intervention studies demonstrate that noni fruit juice provides protection against tobacco smoke-induced DNA damage, blood lipid, and homocysteine elevation, as well as systemic inflammation. Human intervention studies also reveal that noni juice improves joint health, increases physical endurance, increases immune activity, inhibits glycation of proteins, aids weight management, helps maintain bone health in women, helps maintain normal blood pressure, and improves gum health. Further, these studies reveal that noni juice exhibits significant antioxidant activity, more so than other fruit juices, which served as trial placebos. The antioxidant effect of noni juice and its interaction with the immune system and inflammation pathways account for many of the observed health benefits of noni juice. However, several other biological processes of the human body are also positively influenced by noni juice.

Blackberry (Rubus spp.) fruit has been used in traditional herbal medicine as an antimicrobial, anticancer, antidysentery, antidiabetic, and antidiarrheal agent. It also possesses good antioxidant activity due the presence of phytochemicals, including polyphenols, flavonoids, anthocyanins, salicylic acid, and ellagic acid.

Acerola (Malpighia emarginata) fruit contains an abundance of phytochemicals such as carotenoids phenolics, anthocyanins, and flavonoids. This fruit also exerts potential antihyperglycemic, antigenotoxicity, antimicrobial, anticarcinogenic activity, hepatoprotective, and antioxidant activities.

One unique aspect of the of the formulations disclosed in this application is the synergistic inhibition of arginase by the botanical ingredients. Arginase consumes L-arginine, thereby lowering the amount of substrate available to nitric oxide synthase to product NO. A few of the botanical ingredients used in the present formulations exhibit some level of in vitro arginase inhibition alone. But when these ingredients are combined with and tested together, the resulting arginase inhibition is approximately 87.87% greater than expected, as shown in the Working Example below. This indicates a synergistic effect towards arginase inhibition.

With inhibition of arginase, the subsequent increase in NO will also regulate the release of somatostatin which increases pituitary growth hormone release and downstream insulin-like growth factor 1 (IGF-1) production in the liver. This will counter age-related declines in IGF-1, thereby promoting healthy growth and regeneration of tissues and organs, protection of neurons, maintenance of muscle mass in older people, reduction of atherosclerosis burden, and inhibiting oxidative stress.

Gamma-Aminobutyric acid (GABA) is an inhibitory neurotransmitter. Consumption of dietary sources of GABA has been shown to improve sleep quality and shorten sleep latency in human volunteers. Adequate sleep is necessary for maintaining cardiovascular health, immune function, growth hormone production, and many other physiological processes involved in homeostasis.

### Working Example

The methods of making and using the present formulations consider the unique pharmacokinetics, pharmacodynamics, and nutritional properties of the ingredients. Their collective biological activities contribute to the production of NO. In some embodiments, the dietary supplement can be administered via capsule, powder, or liquid delivery format. This type of administration may be consumed daily.

The following discussion is grouped into subheadings. The utilization of the subheadings is for the convenience of the reader only and is not to be construed as limiting in any sense.

### Example

The following example illustrates some of example formulations. These examples are not intended to be limiting in any way. The examples are merely illustrative of the benefits, advantages, and remedial effects of some embodiments of the compositions of the present formulations. The results from ingredient testing have demonstrated unexpected benefits relating to NO production potential by inhibiting arginase at much greater percentages than expected.

### Test One

### Purpose of Experiment and Research Method

The purpose of this experiment was to discover synergistic effects of botanical ingredients on arginase inhibition. Arginase is an enzyme that metabolizes L-arginine. It competes with another enzyme, endothelial nitric oxide synthase (eNOS), to use L-arginine as a substrate. When arginase is inhibited, more L-arginine is available to eNOS, which converts this amino acid to nitric oxide (NO). NO is a biologically active molecule (a reactive neurotransmitter) which may influence health in several ways. The action of NO includes vasorelaxation, which positively influences blood pressure and blood flow. NO also regulates the release of specific hypothalamic peptides, including somatostatin. This, in turn, can increase pituitary growth hormone release and downstream insulin-like growth factor 1 (IGF-1) production in the liver. Mitigating age-related declines in IGF-1 may maintain homeostasis, promotes healthy growth and regeneration of tissues and organs, protects neurons, maintains skeletal muscle mass in older people, reduces atherosclerosis burden, and improves atherosclerotic plaque stability by inhibiting oxidative stress within the plaques.

### Samples and Procedures

The botanical ingredients used in this study were: (i) grape seed extract powder; (ii) freeze-dried blackberry whole fruit powder; (iii) acerola fruit powder; (iv) turmeric powder (v) blueberry fruit powder; and (vi) freeze-dried noni fruit puree from Tahiti.

The samples were evaluated for in vitro arginase inhibition using recombinant human arginase 1. Briefly, human arginase dissolved in bioassay buffer. Samples were also dissolved in deionized water and filtered through 0.45 μm filter. A human liver arginase (ARG1) inhibitor screening kit (item #ab283402, ABCAM, Inc., Cambridge, UK) was used to evaluate the reduction in arginase activity when incubated with varying concentrations of the samples.

The arginase solution was transferred to separate wells of a clear plastic 96 well plate. A well-known and effective inhibitor reference standard, 2-amino-6-boronohexanoic acid (abbreviated as ABH), was included in the assay and added to the appropriate well. Control wells contained no sample, or ABH, and represented uninhibited enzyme activity.

The botanical test samples were added to designated wells. The 96 well plate was then incubated for 5 minutes at 37° C. Afterward, enzyme substrate (containing L-arginine) was added to each well, and the plate incubated at 37° C. for 30 minutes. Next the probe mix (containing sulfuric acid and boric acid) that is used to detect products of the enzymes action on the substrate, was added. The plate was then incubated for 60 minutes at 25° C.

Digital image analysis of final reaction mixtures in the microplate were conducted with a colorimeter and CIE L*a*b* values collected. Enzyme inhibition activity was determined with a* values, specifically the difference between the value of the ABH control of the inhibitor-free control and/or each botanical powder sample. The expected percentages of inhibition are the sum of each individual ingredient's percent inhibition multiplied by the percentage of that ingredient in a blend that contained the other ingredients.

### Results

The results are presented in the graph below. Samples were diluted to a concentration of 200 μg/mL. At a concentration of 200 μg/mL, four of the six samples did not inhibit arginase activity, and the noni fruit powder and grape seed extract respectively exhibited 13.93% and 55.47% arginase inhibition. At a concentration of 33 μg/mL, one of skill in the art would expect the noni fruit powder and grape seed extract to exhibit approximately one-sixth of this arginase inhibition (i.e., 200/33.3) while the remaining four samples continued to exhibit no arginase inhibition. However, the results show that the combination of all six botanical samples, each at a final concentration of 33 μg/mL, 100% of the arginase activity was inhibited. This is 87.87% greater than the expected percent inhibition of 12.04%.

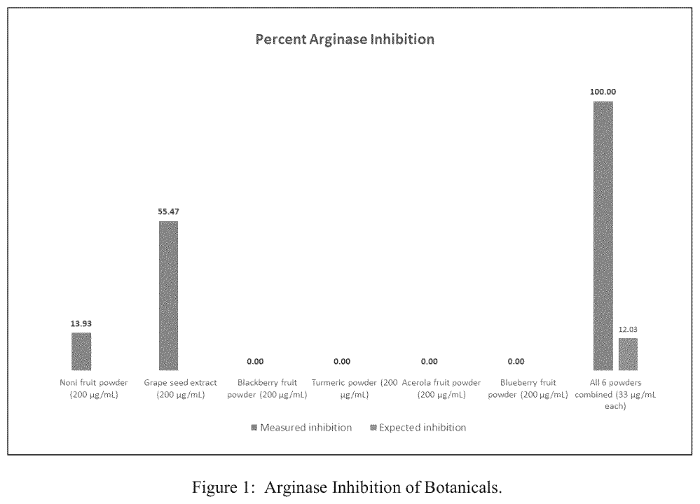

These results clearly demonstrate a synergistic effect. The activity was expected to decrease significantly with the dilution of the noni powder and grape seed extract since the other samples were not active at 200 μg/mL or lower. However, the opposite occurred. Apparently, the combination of phytochemicals providing by the combination of the samples was very effective in limiting arginase activity, much more so than those provided by any single ingredient alone. The test results are illustrated in FIG. 1.

### Applications and Impact

The aging process is a relentless fact of human life and health. Research demonstrates that not all nutrients are adequately obtained from the typical diet. Thus, credible supplementation of certain beneficial micronutrients and phytochemicals is a rational approach in an anti-aging strategy. It is important that the dosages and dose ranges provide wide applicability. In this invention, the primary dosing and ranges are designed to be at broad and sufficient levels to have the desired beneficial anti-aging effects in humans of both genders and of wide age and weight cohorts. The basic classes of micro/phytonutrients to which the components of the present formulation belong are known to enhance growth hormone levels, reduce oxidative damage and inflammation, support immune system function and neuroprotection, and enhance optimal health during aging.

This invention is intended to relate to any form of administration of these formulations in humans including but not limited to tablets, bars, liquids, powders, gummies, lozenges/troches, dissolvable disks, chewables, inhalational forms, injectables, patches, ointments, gels, aerosols, and sticks as well as any transcutaneous, intranasal, intra-cavitary, liposomal, nanotechnology or other delivery system.

What is claimed is:

1. A formulation for an anti-aging dietary supplement that synergistically inhibits arginase while increasing L-arginine and nitric oxide synthase, comprising:
Morinda citrifolia fruit;
Vitis vinifera seed extract;
Malpighia glabra fruit;
Rubus fruticosus fruit;
Curcuma longa root extract which comprises curcumin; and
Vaccinium angustifolium fruit extract.

2. The formulation of claim 1, further comprising an amino acid, wherein the amino acid comprises L-Arginine, L-Citrulline, beta-Alanine, and L-Lysine.

3. The formulation of claim 1, further comprising a carotenoid.

4. The formulation of claim 1, further comprising gamma-aminobutyric acid.

5. The formulation of claim 1, further comprising:
L-Arginine;
L-Citrulline;
L-Lysine;
beta-alanine;
Vitamin B3 as Niacin;
Vitamin B3 as Niacinamide;
Vitamin B6;
Vitamin B12;
chromium;
magnesium;
selenium;
zinc;
a carotenoid; and
gamma-aminobutyric acid.

6. The formulation of claim 1, further comprising, by weight of the formulation:
L-Arginine, L-Citrulline, and L-Lysine in an amount between 120 and 6000 mg;
beta-alanine in an amount between 5 and 250 mg;
Vitamin B3 as Niacin in an amount between 1 and 100 mg;
Vitamin B3 as Niacinamide in an amount between 5 and 1000 mg;
Vitamin B6 in an amount between 0.5 and 25 mg;
Vitamin B12 in an amount between 0.001 and 0.05 mg;
chromium in an amount between 0.005 and 0.25 mg;
magnesium in an amount between 12 and 600 mg;
selenium in an amount between 0.01 and 30 mg;
zinc in an amount between 1.5 and 100 mg;
beta-carotene in an amount between 0.05 and 20 mg;
a carotenoid other than beta-carotene, in an amount between 0.05 and 50 mg;
Vaccinium corymbosum fruit extract in an amount between 1.5 and 75 mg;
Curcuma longa root extract which comprises curcumin in an amount between 1.5 and 125 mg;
Rubus fruticosus fruit in an amount between 1.0 and 75 mg;
Vitis vinifera seed extract in an amount between 1.5 and 125 mg;
Malpighia glabra fruit in an amount between 0.5 and 50 mg;
gamma-aminobutyric acid in an amount between 50 and 2500 mg; and
Morinda citrifolia fruit in an amount between 5 and 500 mg.

7. The formulation of claim 6, further comprising, by weight of the formulation:
L-Arginine in an amount equal to 1200 mg;
L-Citrulline in an amount equal to 1218.27 mg;
L-Lysine in an amount equal to 1218.27 mg;
beta-alanine in an amount equal to 50 mg;
Vitamin B3 as Niacin in an amount equal to 5.05 mg;
Vitamin B3 as Niacinamide in an amount equal to 20.3 mg;
Vitamin B6 in an amount equal to 6.25 mg;
Vitamin B12 in an amount equal to 0.01 mg;
chromium in an amount equal to 0.27 mg;
magnesium in an amount equal to 250 mg;
selenium in an amount equal to 20 mg;
zinc in an amount equal to 50 mg;
beta-carotene in an amount equal to 0.434 mg;
a carotenoid other than beta-carotene, in an amount equal to 1.8 mg;
Vaccinium corymbosum fruit extract in an amount equal to 25 mg;
Curcuma longa root extract which comprises curcumin in an amount equal to 25 mg;
Rubus fruticosus fruit in an amount equal to 15 mg;
Vitis vinifera seed extract in an amount equal to 25 mg;
Malpighia glabra fruit in an amount equal to 5 mg;
gamma-aminobutyric acid in an amount equal to 505.05 mg; and
Morinda citrifolia fruit in an amount equal to 65 mg.

8. The formulation of claim 1, further comprising, by weight of the formulation:
L-Arginine, L-Citrulline, and L-Lysine in an amount between 120 and 6000 mg;
beta-alanine in an amount between 5 and 250 mg;
Vitamin B3 as Niacinamide in an amount between 5 and 1000 mg;
Vitamin B6 in an amount between 0.5 and 25 mg;
Vitamin B12 in an amount between 0.001 and 1.5 mg;
chromium in an amount between 0.005 and 0.5 mg;
magnesium in an amount between 12 and 600 mg;
selenium in an amount between 0.01 and 50 mg;
zinc in an amount between 1.5 and 125 mg;
beta-carotene in an amount between 0.05 and 20 mg;
a carotenoid other than beta-carotene, in an amount between 0.05 and 50 mg;
Vaccinium corymbosum fruit extract in an amount between 1.5 and 75 mg;
Curcuma longa root extract which comprises curcumin in an amount between 1.5 and 125 mg;
Rubus fruticosus fruit in an amount between 1.0 and 75 mg;
Vitis vinifera seed extract in an amount between 1.5 and 125 mg;
Malpighia glabra fruit in an amount between 0.5 and 50 mg;
gamma-aminobutyric acid in an amount between 50 and 2500 mg; and
Morinda citrifolia fruit in an amount between 5 and 500 mg.

9. The formulation of claim 8, further comprising, by weight of the formulation:
L-Arginine in an amount equal to 1400 mg;
L-Citrulline in an amount equal to 1015.22 mg;
L-Lysine in an amount equal to 1218.27 mg;
beta-alanine in an amount equal to 50.76 mg;
Vitamin B3 as Niacinamide in an amount equal to 25.38 mg;
Vitamin B6 in an amount equal to 6.25 mg;
Vitamin B12 in an amount equal to 0.36 mg;
chromium in an amount equal to 0.15 mg;
magnesium in an amount equal to 250 mg;
selenium in an amount equal to 50 mg;
zinc in an amount equal to 125 mg;
beta-carotene in an amount equal to 0.434 mg;
a carotenoid other than beta-carotene, in an amount equal to 1.8 mg;
Vaccinium corymbosum fruit extract in an amount equal to 25 mg;
Curcuma longa root extract which comprises curcumin in an amount equal to 25 mg;
Rubus fruticosus fruit in an amount equal to 15 mg;
Vitis vinifera seed extract in an amount equal to 25 mg;
Malpighia glabra fruit in an amount equal to 5 mg;
gamma-aminobutyric acid in an amount equal to 505.05 mg; and
Morinda citrifolia fruit in an amount equal to 65 mg.

10. A formulation for an anti-aging dietary supplement that synergistically inhibits arginase while increasing L-arginine and nitric oxide synthase, comprising:
Morinda citrifolia fruit;
Vitis vinifera seed extract;
Malpighia glabra fruit;
Rubus fruticosus fruit;
Garcinia mangostana L. fruit extract;
Curcuma longa root extract which comprises curcumin; and
Vaccinium angustifolium fruit extract.

11. The formulation of claim 10, further comprising an amino acid, wherein the amino acid comprises L-Arginine, L-Citrulline, beta-Alanine, and L-Lysine.

12. The formulation of claim 10, further comprising a carotenoid.

13. The formulation of claim 10, further comprising Garcinia mangostana L.

14. The formulation of claim 10, further comprising GABA.

15. The formulation of claim 10, further comprising:
L-Arginine;
L-Citrulline;
L-Lysine;
beta-alanine;
Vitamin B3 as Niacin;
Vitamin B3 as Niacinamide;
Vitamin B6;
Vitamin B12;
magnesium;
zinc;
a carotenoid; and
gamma-aminobutyric acid.

16. The formulation of claim 10, further comprising, by weight of the formulation:
L-Arginine, L-Citrulline, and L-Lysine in an amount between 120 and 6000 mg;
beta-alanine in an amount between 5 and 250 mg;
Vitamin B3 as Niacin in an amount between 1 and 100 mg;
Vitamin B3 as Niacinamide in an amount between 5 and 1000 mg;
Vitamin B6 in an amount between 0.5 and 25 mg;
Vitamin B12 in an amount between 0.001 and 0.05 mg;
chromium in an amount between 0.005 and 0.25 mg;
magnesium in an amount between 12 and 600 mg;
selenium in an amount between 0.01 and 30 mg;
zinc in an amount between 1.5 and 100 mg;
beta-carotene in an amount between 0.05 and 20 mg;
a carotenoid other than beta-carotene, in an amount between 0.05 and 50 mg;
Vaccinium corymbosum fruit extract in an amount between 1.5 and 75 mg;
Curcuma longa root extract which comprises curcumin in an amount between 1.5 and 125 mg;
Rubus fruticosus fruit in an amount between 1.0 and 75 mg;
Vitis vinifera seed extract in an amount between 1.5 and 125 mg;
Malpighia glabra fruit in an amount between 0.5 and 50 mg;
gamma-aminobutyric acid in an amount between 50 and 2500 mg; and
Morinda citrifolia fruit in an amount between 5 and 500 mg.

17. The formulation of claim 16, further comprising, by weight of the formulation:
L-Arginine in an amount equal to 1200 mg;
L-Citrulline in an amount equal to 1218.27 mg;
L-Lysine in an amount equal to 1218.27 mg;
beta-alanine in an amount equal to 50 mg;
Vitamin B3 as Niacin in an amount equal to 5.05 mg;
Vitamin B3 as Niacinamide in an amount equal to 20.3 mg;
Vitamin B6 in an amount equal to 6.25 mg;
Vitamin B12 in an amount equal to 0.01 mg;
chromium in an amount equal to 0.27 mg;
magnesium in an amount equal to 250 mg;
selenium in an amount equal to 20 mg;
zinc in an amount equal to 50 mg;
beta-carotene in an amount equal to 0.434 mg;
a carotenoid other than beta-carotene, in an amount equal to 1.8 mg;
Vaccinium corymbosum fruit extract in an amount equal to 25 mg;
Curcuma longa root extract which comprises curcumin in an amount equal to 25 mg;
Rubus fruticosus fruit in an amount equal to 15 mg;
Vitis vinifera seed extract in an amount equal to 25 mg;
Malpighia glabra fruit in an amount equal to 5 mg;
gamma-aminobutyric acid in an amount equal to 505.05 mg; and
Morinda citrifolia fruit in an amount equal to 65 mg.

18. The formulation of claim 10, further comprising, by weight of the formulation:
L-Arginine, L-Citrulline, and L-Lysine in an amount between 120 and 6000 mg;
beta-alanine in an amount between 5 and 250 mg;
Vitamin B3 as Niacin in an amount between 0.1 and 20 mg;
Vitamin B6 in an amount between 0.01 and 5 mg;
Vitamin B12 in an amount between 0.001 and 0.5 mg;
magnesium in an amount between 0.5 and 15 mg;
zinc in an amount between 1.5 and 75 mg;
beta-carotene in an amount between 0.05 and 1.5 mg;
a carotenoid other than beta-carotene, in an amount between 0.05 and 10 mg;
Vaccinium corymbosum fruit extract in an amount between 1.5 and 75 mg;
Curcuma longa root extract which comprises curcumin in an amount between 2.5 and 125 mg;
Rubus fruticosus fruit in an amount between 1.5 and 75 mg;
Vitis vinifera seed extract in an amount between 2.5 and 125 mg;
Malpighia glabra fruit in an amount between 1.5 and 75 mg;
Garcinia mangostana L. in an amount between 0.01 to 35 mg;
gamma-aminobutyric acidin an amount between 50 and 2500 mg; and
Morinda citrifolia fruit in an amount between 10 and 500 mg.

19. The formulation of claim 18, further comprising, by weight of the formulation:
L-Arginine in an amount equal to 1200 mg;
L-Citrulline in an amount equal to 1218.27 mg;
L-Lysine in an amount equal to 1218.27 mg;
beta-alanine in an amount equal to 50 mg;
Vitamin B3 as Niacin in an amount equal to 1.08 mg;
Vitamin B6 in an amount equal to 0.111 mg;
Vitamin B12 in an amount equal to 0.003 mg;
magnesium in an amount equal to 3 mg;
zinc in an amount equal to 15 mg;
beta-carotene in an amount equal to 0.434 mg;
a carotenoid other than beta-carotene, in an amount equal to 1.8 mg;
Vaccinium corymbosum fruit extract in an amount equal to 30 mg;
Curcuma longa root extract which comprises curcumin in an amount equal to 25 mg;
Rubus fruticosus fruit in an amount equal to 15 mg;
Vitis vinifera seed extract in an amount equal to 25 mg;
Malpighia glabra fruit in an amount equal to 5 mg;
Garcinia mangostana L. in an amount equal to 30 mg;
gamma-aminobutyric acid in an amount equal to 505.05 mg; and
Morinda citrifolia fruit in an amount equal to 65 mg.

20. A method for administering an anti-aging dietary supplement that synergistically inhibits arginase while increasing L-arginine and nitric oxide synthase, comprising:
combining Morinda citrifolia fruit; Vitis vinifera seed extract; Malpighia glabra fruit; Rubus fruticosus fruit; Curcuma longa root extract which comprises curcumin; and Vaccinium angustifolium fruit extract, to form a dietary supplement; and
administering the dietary supplement daily to inhibit arginase activity.
